(19) United States
(12) Patent Application Publication
(10) Pub. No.: US 2007/0269808 A1
(43) Pub. Date: Nov. 22, 2007

(54) Wnt receptor compositions and methods

(76) Inventors: Purnima Bhanot, Baltimore, MD (US); Marcel Brink, Alphen a/d Rijn (NL); Cindy H. Samos, Palo Alto, CA (US); Yanshu Wang, Baltimore, MD (US); Jen-Chih Hsieh, Baltimore, MD (US); Deborah Andrew, Baltimore, MD (US); Jeremy Nathans, Baltimore, MD (US); Roeland Nusse, Stanford, CA (US)

Correspondence Address: ROPES & GRAY LLP;PATENT DOCKETING 39/41, ONE INTERNATIONAL PLACE, BOSTON, MA, 02110-2624, US

(21) Appl. No.: 11/370,191

(22) Filed: Mar. 6, 2006

Related U.S. Application Data
(63) Continuation of application No. 10/152,548, filed on May 21, 2002, now abandoned.
Application No. 10/152,548 is a continuation of application No. 08/832,340, filed on Apr. 11, 1997, now abandoned.
(60) Provisional application No. 60/015,307, filed on Apr. 12, 1996.

(51) Int. Cl. C12Q 1/68; C07H 21/04; C07K 1/00; C12N 15/00

(52) U.S. Cl. 435/6; 435/320.1; 530/350; 536/23.5

(57) ABSTRACT

Wnt receptor compositions and methods of use are disclosed. In particular, methods using Wnt receptors, such as Dfz2, in screens for compounds which modulate the binding of a Wnt polypeptide to a Wnt receptor.

The present invention claims priority under 35 USC 120 to U.S. provisional patent application 60/015,307.

This work was supported in part by a grant from the National Cancer Institute. Accordingly, the United States Government has certain rights in this invention.

### FIELD OF THE INVENTION

The present invention relates to Wnt receptor compositions and therapeutic and diagnostic methods related thereto.

### REPERENCES

Auffray, C., and Rougeon, F., Eur. J. Biochem. 107:303-314 (1980).
Ausubel, F. M., et al., Current Protocols in Molecular Biology, John Wiley and Sons, Inc., Media, Pa. (1988).
Barbas, C. F., e t al., Proc. Natl. Acad. Sci. USA 89 (10):4457 (1992)
Bunin, B. A., et al., Proc. Natl. Acad. Sci. USA 91:4708 (1994).
Bunin, B. A. and Ellman, J. A., J. Am. Chem. Soc. 114:10997 (1992).
Chan, S. D. H., et al., J. Biol. Chem. 267:25202 (1992).
Cole, A., et al., J. Neurochem. 55:1920-1927 (1990).
Couso, J. P. and Martinez Arias, A., Cell 79:259-272 (1994).
Dooley, C. T., et al., Proc. Natl. Acad. Sci. USA 90(22):10822 (1993a).
Dooley, C. T., et al., Life Sci. 52(18):1509 (1993b).
Ecker, D. J., et al., Nuc. Acids Res. 21(8):1853 (1993).
Eichler, J., et al., Biochemistry 32(41):11035 (1993).
Eisenberg, L. M., et al., Dev Biol. 154(1):73-83 (1992).
Furka, A., et al., Int. J. Pept. Protein Res. 37:487-493 (1991).
Gennaro, A. R., Ed., Remington'S Pharmaceutical Sciences (18th ed., Mack Publishing Co., Easton Pa. (1990)).
Graba, Y., et al., Development 121(1):209-218 (1995).
Harlow, E., et al., Antibodies: A Laboratory Manual, Cold Spring Harbor Laboratory Press (1988).
Houghten, R. A., Proc. Natl. Acad. Sci. USA 85:5131-5135 (1985).
Houghten, R. A., Current Biology 4:564 (1994).
Houghten, R. A., et al., BioTechniques 4:522-528 (1986).
Houghten, R. A., et al., Nature 354:84-86 (1991).
Houghten, R. A., et al., BioTechniques 13:412-421 (1992).
Kramer, A., et al., Pept. Res. 6(6):314 (1993).
Lam, K. S., et al., Nature (London) 354:82-84 (1991).
Lam, K. S., et al., Bioorg. Med. Chem. Lett. 3:419-424 (1993).
Maniatis, T., et al., in Molecular Cloning: A Laboratory Manual, Cold Spring Harbor Press, Cold Spring Harbor, N.Y. (1982).
Mullis, K. B., U.S. Pat. No. 4,683,202, issued 28 Jul. 1987.
Mullis, K. B., et al., U.S. Pat. No. 4,683,195, issued 28 Jul. 1987.
Nusse, R., and Varmus, H. E., Cell 69(7):1073-1087 (1992).
O'Connell, P. and Rosbash, M., Nuc. Acids Res. 12:5495-5513 (1984).
Oda, H., et al., J. Cell Biol. 121:1133-1140 (1993).
Ohlmayer, M. H., et al., Proc Nat Acad Sci, USA, 90(23):10922 (1993).
Pearson, W. R., Methods in Enzymology 183:63-98 (1990).
Pearson, W. R. and Lipman, D. J., PNAS 85:2444-2448 (1988).
Peifer, M., et al., Development 120:369-380 (1994).
Pinilla, C., et al., Biotechniques 13(6):901 (1992).
Pinilla, C., et al., Gene 128(1):71 (1993).
Riggleman, B., et al., Cell 63:549-560 (1990).
Russell, J., et al., Development 115:475-485 (1992).
Sambrook, J., et al., Molecular Cloning: A Laboratory Manual 2 Ed., Vol. 2., Cold Spring Harbor: Cold Spring Harbor Laboratory Press (1989).
Sebestyen, F., et al., Bioorg. Med. Chem. Lett. 3:413-418 (1993)
Saffen, D., et al., Proc Nat Acad Sci, USA, 85:7795 (1988).
Van Leeuwen, F., et al., Nature 368:342-344 (1994).
Virgilio, A. A., and Ellman, J. A., J. Am. Chem. Soc. 116:11580 (1994).
Yanagawa, S., et al., Genes & Dev. 9: 1087-1097 (1995).
Zuckermann, R. N., et al., Int. J. Pept. Protein Res. 40:498-507 (1992).

### BACKGROUND OF THE INVENTION

Wnt genes encode secreted proteins involved in cell-to-cell signaling. Wnt genes play important growth controlling roles, in particular in the mammary gland, and act as oncogenes in mouse mammary tumors. Little is known about the mechanism of action of Wnt products, in part because Wnt receptors have until now remained unidentified.

### SUMMARY OF THE INVENTION

In one aspect, the present invention includes an isolated nucleic acid molecule encoding a Wnt receptor (WntR) polypeptide. In a general embodiment, the WntR polypeptide has an amino acid sequence that is greater than about 90% identical to the amino acid sequence of at least one of the following Wnt receptors: Dfz1, Dfz2, Rfz1, Rfz2, Hfz3, Hfz4, Hfz5, Mfz3, Mfz4, Mfz5, Mfz6, Mfz7, Mfz8, and Cfz1. In a related embodiment, the Wnt receptor has an amino acid sequence that is more than about 90% identical to an amino acid sequence contained SEQ ID NO:2, SEQ ID NO:4, SEQ ID NO:6, SEQ ID NO:8, SEQ ID NO:10, SEQ ID NO:12, SEQ ID NO:14 or SEQ ID NO:16. Exemplary WntRs described herein have an amino acid sequence that is more than about 90% identical to one of the following sequences: SEQ ID NO:2, SEQ ID NO:4, SEQ ID NO:6, SEQ ID NO:8, SEQ ID NO:10, SEQ ID NO:12, SEQ ID NO:14 or SEQ ID NO:16. In preferred embodiments, the WntR polypeptide has an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14 and SEQ ID NO:16.

Examples of nucleic acid molecules encoding Wnt receptor polypeptides are provided herein as SEQ ID NO:1, SEQ ID NO:3, SEQ ID NO:5, SEQ ID NO:7, SEQ ID NO:9, SEQ ID NO:11, SEQ ID NO:13 and SEQ ID NO:15. Preferred embodiments are human Wnt polynucleotides. An exemplary human Wnt polynucleotide has the sequence presented as SEQ ID NO:9.

The invention further includes fragments of polynucleotides encoding full-length WntR, where the fragments are of sufficient length to hybridize selectively with a Wnt polynucleotide sequence or complement thereof, such as a sequence selected from the group consisting of SEQ ID NO:1, SEQ ID NO:3, SEQ ID NO:5, SEQ ID NO:7, SEQ ID NO:9, SEQ ID NO:11, SEQ ID NO:13 and SEQ ID NO:15. Such fragments are at least 15, preferably at least about 18, 21 or 24, nucleotides in length.

In another aspect, the invention includes an isolated Wnt receptor polypeptide. In a general embodiment, the polypeptide has an amino acid sequence that is more than about 90% identical to the amino acid sequence of a Wnt receptor selected from the group consisting of Dfz1, Dfz2, Rfz1, Rfz2, Hfz3, Hfz4, Hfz5, Mfz3, Mfz4, Mfz5, Mfz6, Mfz7, Mfz8, and Cfz1. In a related embodiment, the polypeptide has an amino acid sequence that is more than about 90% identical to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14 and SEQ ID NO:16. In another related embodiment, the polypeptide sequence is selected from the group consisting of SEQ ID NO:2, SEQ ID NO:4, SEQ ID NO:6, SEQ ID NO:8, SEQ ID NO:10, SEQ ID NO:12, SEQ ID NO:14 and SEQ ID NO:16.

Preferred embodiments are human Wnt polypeptides. An exemplary human Wnt polypeptide has the sequence presented as SEQ ID NO:10.

The invention further includes peptide fragments derived from a full-length WntR polypeptide, where the fragments contain a region of at least seven, preferably at least ten, consecutive amino acids, and where the region has at least about an 80% identity with the residues of a corresponding region of a polypeptide having a sequence selected from the group consisting of SEQ ID NO:2, SEQ ID NO:4, SEQ ID NO:6, SEQ ID NO:8, SEQ ID NO:10, SEQ ID NO:12, SEQ ID NO:14 and SEQ ID NO:16.

Also included in the invention are antibodies, both monoclonal and polyclonal, specifically-immunoreactive with Wnt receptor polypeptides. Such antibodies may be produced using standard methods (Harlow).

The invention also includes a method of identifying a compound capable of affecting binding of a Wnt polypeptide to a Wnt receptor polypeptide. The method includes (i) contacting such a Wnt receptor polypeptide with a selected Wnt polypeptide, in the presence and absence of a test compound, (ii) measuring the effect of the test compound on the extent of binding between the Wnt polypeptide and the Wnt receptor polypeptide, and (iii) identifying said compound as effective if its measured effect on the extent of binding is above a threshold level. In a general embodiment, the method includes an additional step (iv) comprising preparing a pharmaceutical preparation of a compound identified as effective to alter binding of a Wnt polypeptide to a WntR polypeptide.

In one embodiment, the threshold is a 2-fold or greater inhibition of binding. In another embodiment, the threshold is a 2-fold or greater potentiation of binding. Examples of suitable Wnt polypeptides include wingless (Wg); examples of suitable Wnt receptor polypeptides include Dfz2 (e.g., SEQ ID NO:2).

The test compound may be effective to inhibit binding between the Wnt polypeptide and the Wnt receptor or to displace the Wnt polypeptide from the Wnt receptor polypeptide. In one embodiment, the Wnt receptor polypeptide is expressed on the surface of a cell (e.g., Drosophila Sneider 2 (S2) cell) transformed with an expression vector encoding said receptor (e.g., Dfz2).

In another embodiment, the Wnt receptor polypeptide is an N-terminal portion of a full-length Wnt receptor polypeptide, the N-terminal portion including the cysteine-rich amino-terminal domain. In one embodiment, the N-terminal portion is part of a fusion with, e.g., the constant domain of human IgG.

These and other objects and features of the invention will become more fully apparent when the following detailed description is read in conjunction with the accompanying drawings.

### BRIEF DESCRIPTION OF THE FIGURES

FIG. 1 shows a sequence comparison of Dfz1 and Dfz2′.

FIG. 2 shows hydropathy profiles of mammalian and nematode frizzled homologs.

FIG. 3 shows a computer-generated image of the expression of DFz2 during Drosophila development evaluated by Northern blot.

FIG. 4 is a computer-generated image showing that transfection of DFz2 into S2 cells confers a response to Wg protein.

FIG. 5 is a computer-generated image made using confocal immunomicroscopy showing binding of Wg protein to Dfz-2 transfected cells.

FIG. 6 is a computer-generated image showing the binding of metabolically labeled Wg protein to a Dfz-2/Ig fusion protein.

### DETAILED DESCRIPTION OF THE INVENTION

I. Definitions

An “isolated” polypeptide, polynucleotide or antibody refers to such a polypeptide, polynucleotide or antibody that has been at least partially purified away from other cellular material.

A polynucleotide sequence or fragment is “derived from” another polynucleotide sequence or fragment when it contains the same sequence of nucleotides as are present in the sequence or fragment from which it is derived. For example, a bacterial plasmid contains an insert “derived from” a selected human gene if the sequence of the polynucleotides in the insert is the same as the sequence of the polynucleotides in the selected human gene.

Similarly, a polypeptide sequence or fragment is “derived from” another polypeptide sequence or fragment when it contains the same sequence of amino acids as are present in the sequence or fragment from which it is derived. A polypeptide “derived from” a nucleic acid is a polypeptide encoded by that nucleic acid. For example, a Wnt receptor polypeptide derived from the human genome (also termed “human Wnt receptor polypeptide” or “hWntR”) is a polypeptide encoded by an mRNA (or corresponding cDNA) transcribed from a human Wnt receptor gene.

Percent (%) identity, with respect to two amino acid sequences, refers to the % of residues that are identical in the two sequences when the sequences are optimally aligned and no penalty is assigned to “gaps”. In other words, if a gap needs to be inserted into a first sequence to optimally align it with a second sequence, the % identity is calculated using only the residues that are paired with a corresponding amino acid residue (i.e., the calculation does not consider residues in the second sequences that are in the “gap” of the first sequence). Optimal alignment is defined as the alignment giving the highest % identity score. Such alignments can be preformed as described herein using the “GENEWORKS” program. Alternatively, alignments may be performed using the local alignment program LALIGN with a ktup of 1, default parameters and the default PAM. The LALIGN program is found in the FASTA version 1.7 suite of sequence comparison programs (Pearson and Lipman, 1988; Pearson, 1990; program available from William R. Pearson, Department of Biological Chemistry, Box 440, Jordan Hall, Charlottesville, Va.).

A full-length Wnt receptor (WntR) polypeptide is defined herein as a polypeptide that is a member of the frizzled protein family, encodes a full-length protein, and has at least about a 90% identity with one or more of the following sequences: SEQ ID NO:2, SEQ ID NO:4, SEQ ID NO:6, SEQ ID NO:8, SEQ ID NO:10, SEQ ID NO:12, SEQ ID NO:14 and SEQ ID NO:16.

II. Overview of the Invention

The present invention is based on the discovery of a set of novel members of the vertebrate frizzled family of polarity genes, and on the recognition that the frizzled family of polarity genes encodes the receptors for the Wnt family of proteins. The invention is further enhanced by the recognition that the full-length sequence of each member of the frizzled protein family generally shares a substantially greater degree of homology with the full-length sequences of corresponding frizzled proteins in other species (typically about 80% to >95%) than it does with the full-length sequences of other members of the frizzled protein family in the same species (typically about 30% to 60%). Different members of the frizzled family, however, do contain regions within the coding sequences that have high degrees of homology (up to 90% or more) with one another. This feature, combined with similar sizes and hydrophobicity profiles, facilitates the identification of novel members of the frizzled gene family.

Discoveries described herein enable a number of uses and application of the present invention. These uses and applications are exemplified and discussed in detail below.

III. Identification of Dfz2 as the Wg Receptor

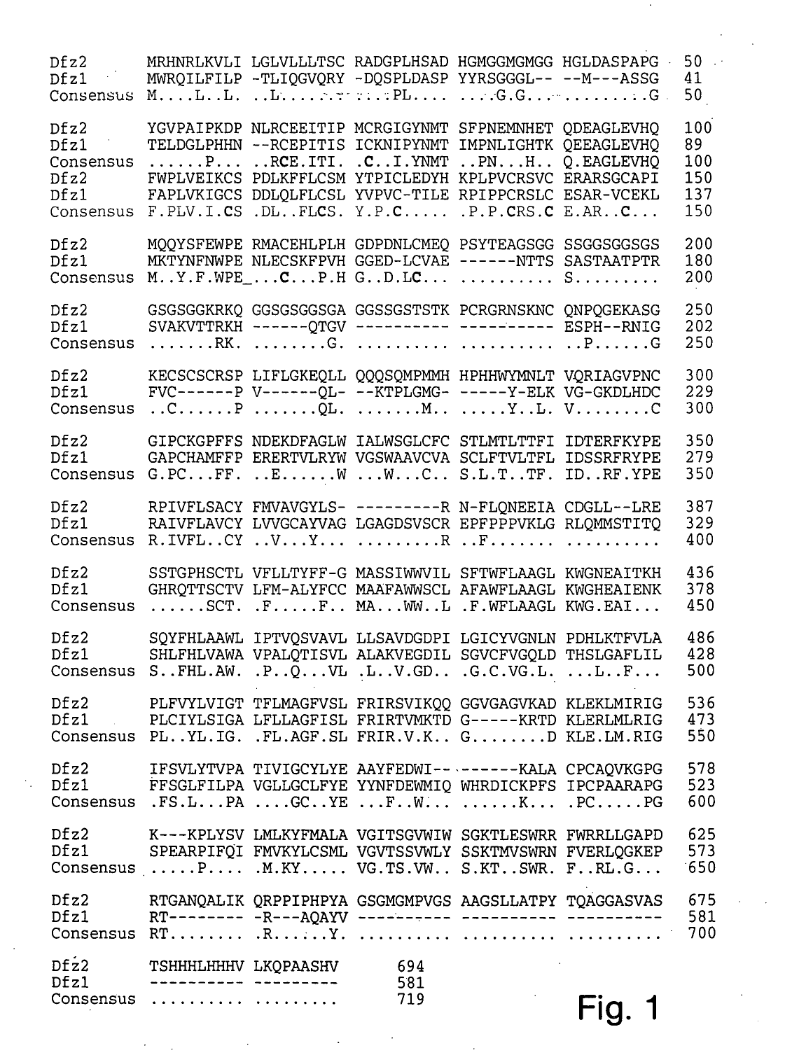

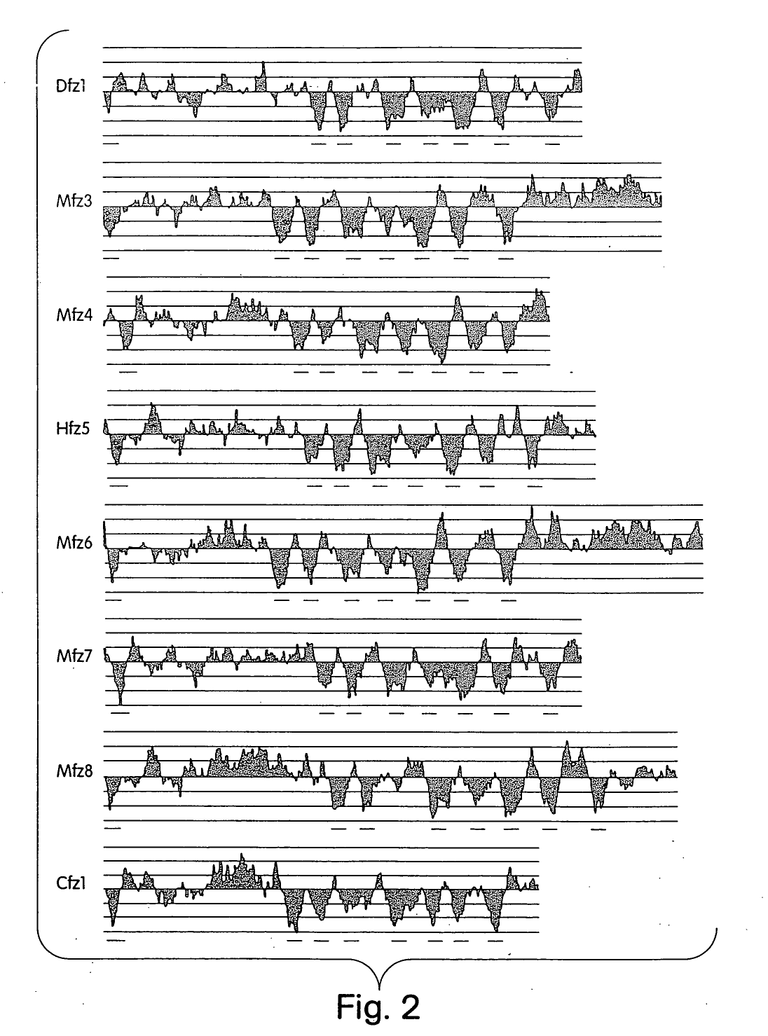

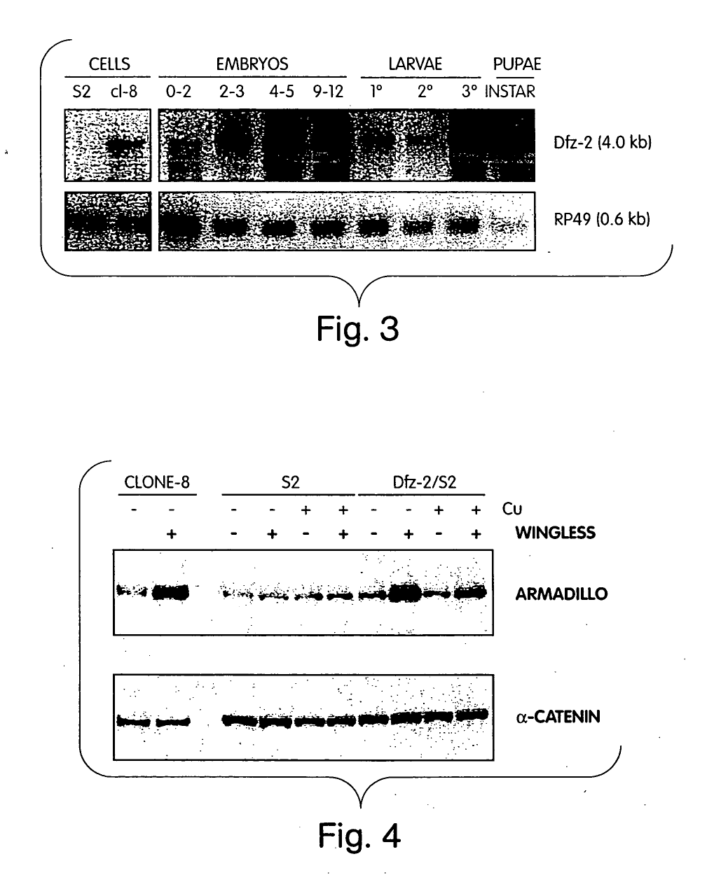

Experiments performed in support of the present invention and described in Examples 1-6, below, indicate that Drosophila frizzled gene 2 (Dfz2) is a receptor for wingless (Wg). Example 1 details the cloning of Dfz2, the sequence of which is illustrated in FIG. 1. Hydrophobicity profiles of additional frizzled family members isolated as part of the present invention are shown in FIG. 2. Their sequences are presented in the Sequence Listing. Example 2 describes in situ hybridization experiments to determine the pattern of Dfz2 expression. Example 3 describes Northern analyses (FIG. 3) showing that Dfz2 is expressed throughout development.

In Example 4, below, Drosophila Sneider 2 (S2) cells were transformed with a Dfz2 expression vector and the effects of the Dfz2 ligand, Wg, were assessed by measuring the levels of armadillo (Arm) protein in response to Wg application (Peifer, et al., 1994; Riggleman, et al., 1990; Van Leeuwen, et al., 1994). The results, shown in FIG. 4, demonstrate that all four Dfz2-transfected S2 cell lines tested showed increased armadillo signal in response to Wg, whereas no such effect was observed with untransfected S2 cells. These results demonstrate that Dfz2 acts as a signal transducing molecule for Wg, consistent with it being a receptor for Wg.

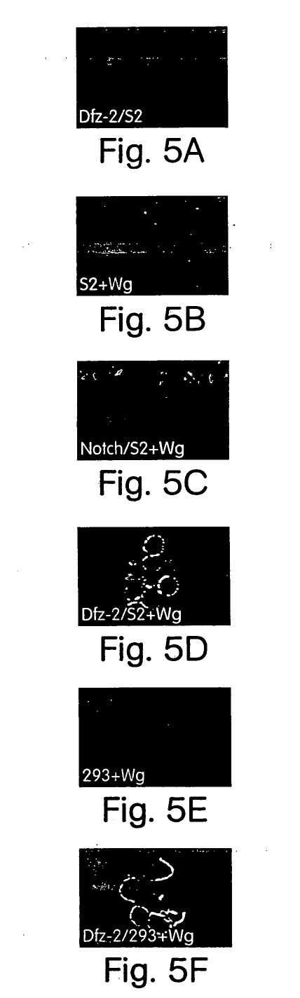

Further support is provided by immunohistochemical analyses described in Example 5. These experiments were designed to address whether Wg was capable of binding to the Dfz2-transfected cells. Dfz2-transfected and nontransfected cells were exposed to medium containing Wg protein, washed, stained with an anti-Wg antiserum and a labelled secondary antibody, and imaged using a confocal microscope. Exemplary images, shown in FIGS. 5A-5F, demonstrate that approximately 80% of Dfz2-transfected S2 cells exposed to Wg protein stained brightly (FIG. 5D) whereas Dfz2-transfected cells in the absence of Wg protein (FIG. 5A) as well as non transfected S2 cells (FIG. 5B) did not. The ability of Wg to bind was also tested in human 293 cells, which are heterologous to the Dfz2 protein. As shown in FIG. 5F, about 10-20% of the transfected cells remained positive, similar to the transfection efficiency of 293 cells. Since 293 cells are of human origin, these results indicate that Wg binds to Dfz2 itself, rather than to a molecule whose expression is induced by Dfz2.

The binding of Wg protein to Dfz2 was further confirmed using a fusion protein containing the cysteine-rich amino-terminal domain of Dfz2, linked to the constant domain of human IgG, as described in Example 6. The fusion protein or IgG control was added to conditioned medium from normal S2 cells, or S2 cells producing Wg (HS-wg/S2), which had been metabolically-labeled with [³⁵S] cysteine and methionine.

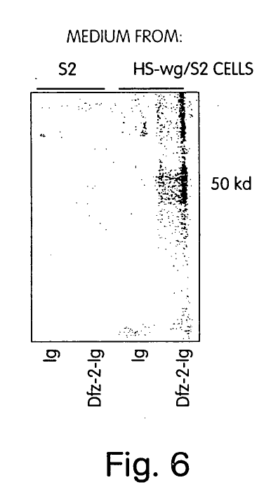

The fusion proteins and possible complexes were then isolated and analyzed by gel electrophoresis and fluorography (FIG. 6). Two bands of approximately 52 kd (the size of Wg) were detected in the lane with the Dfz2-Ig fusion added to the medium of HS-wg/S2 cells.

The above results taken together, particularly the observations that (i) Wg binds to DFz2, and (ii) the binding leads to a biological response, strongly support the role of Dfz2 as the receptor for the Wg protein.

IV. Novel Frizzled Family Members Identified in Vertebrates

Experiments performed in support of the present invention have further resulted in the identification of at least six novel frizzled family members, termed Wnt receptors (WntRs) herein, in human and mouse. This brings the total number of frizzled-like sequences identified in mammalian genomes to 8, since two (Rfz1 and Rfz2) were previously cloned from rat (Chan, et al., 1992). The six novel genes include Mfz3, Mfz4, Mfz6, Mfz7, and Mfz8, as well as human sequences Hfz3, Hfz5 and Hfz7. A sequence 95% identical over 143 amino acids to Hfz5 was PCR-amplified (Mullis, 1987; Mullis, et al., 1987) from mouse genomic DNA using Hfz5-specific primers, suggesting that an Mfz5 gene exists as well. The DNA and translated amino acid sequences of these 6 family members are provided in the Sequence Listing, along with the sequence of a novel family member isolated from C. elegans (Cfz1). The hydrophobicity profiles of these sequences are presented in FIG. 2. These profiles, along with the sequences of regions that are conserved among different frizzled family members, are used in determining whether a polypeptide sequence is a member of the frizzled gene family. According to the present invention, member of this family are considered to be Wnt receptors.

Using the guidance herein, one of skill in the art can isolate additional members of the frizzled gene family. In particular, probes homologous to regions conserved among the various family members can be designed and used to probe cDNA or genomic DNA libraries. Hybridization techniques for isolating related sequences are known in the art (e.g., Ausubel, et al., 1989; Sambrook, et al.). These techniques employ “selective hybridization” to detect sequences similar to the probe sequence with high specificity and low background. Selective hybridization is achieved by increasing the stringency of the wash following hybridization, which can in turn be accomplished through the manipulation of salt concentration and/or temperature of the wash buffer (typically 0.1-10×SSC, 0.1 SDS). Lower salt and higher temperatures result in more stringent hybridization conditions.

Selective hybridization conditions are typically determined empirically for each new probe using several rounds of washes at increasing wash temperatures. The filter or membrane containing the test sequences is evaluated for probe signal after each wash. Wash conditions that give a strong positive signal for a control probe sequence with little or no background signal for unrelated sequences are termed “selective hybridization conditions. Oligonucleotides as small as 16-18 nucleic acids in length can be used to identify similar sequences using hybridization. Of course, the method works well for longer polynucleotides as well.

Alternatively or in addition, PCR primers corresponding to such conserved regions may be designed and used to isolate additional sequences from any suitable source of DNA, including libraries and reverse transcription (RT)-generated cDNA samples.

Exemplary WntR polynucleotide sequences are provided herein as sequences SEQ ID NO:1, SEQ ID NO:3, SEQ ID NO:5, SEQ ID NO:7, SEQ ID NO:9, SEQ ID NO:11, SEQ ID NO:13 and SEQ ID NO:15. These or other WntR polynucleotides may be used to recombinantly produce WntR polypeptides using standard expression vectors known in the art. Many such vectors are available commercially (from Invitrogen, Clontech, or Stratagene). Alternatively, WntR polypeptides may be isolated from native sources or produced synthetically.

V. Wnt Genes and Proteins

Wg in Drosophila is part of larger gene family (Eisenberg, et al., 1992; Graba, et al., 1995; Russell, et al., 1992) of Wnt genes. At least 3 homologous genes have been identified in Drosophila, and over 10 Wnt genes have been identified in most vertebrates (Nusse and Varmus, 1992). According to the present invention, the products of these genes are the ligands for receptors encoded by the large family of fz-like genes in vertebrates. Determination of which Wnt gene products are specific to which Wnt receptor may be performed by one of skill in the art following the teachings of the present specification.

All members of the Wnt family encode secreted proteins that act as cell-cell signaling molecules. Wnt genes play an important role in the control of cell growth, particularly in the mammary gland, and can act as oncogenes in mouse mammary tumors. The proteins contain a signal sequence, one or several N-linked glycosylation sites and many cysteine residues. The product of the mouse Wnt-1 gene has been studied most extensively. If Wnt-1 is overexpressed in various cell lines, the protein enters the secretory pathway. The protein can be detected in protease resistant structures, presumably secretory vesicles, and contains carbohydrate structures at several N-linked glycosylation sites. It is thus generally assumed that the Wnt-1 protein is secreted from cells, although extracellular forms of the protein have been difficult to detect. In addition, most of the intracellular Wnt-1 protein made in transfected cells is incompletely glycosylated (it remains sensitive to endoglycosidase H) and has probably not traversed the Golgi apparatus. Moreover, much of the Wnt-1 protein becomes associated with the resident ER protein BiP, indicating that it is incorrectly folded.

In spite of these difficulties, it has been shown that Wnt-1 overproduction leads to secretion of modest amounts of extracellular protein. The secreted forms have undergone more extensive glycosylations, and may bind to the cell surface or to the extracellular matrix.

VI. Role of Wnt in Cancer

Members of the Wnt gene family are important regulators of mammary cell growth. Indeed, Wnt genes owe their discovery to their role as oncogenes in mouse mammary cancer: previous experiments which examined the sequence around integration sites for Mouse Mammary Tumor Virus (MMTV) DNA showed that many tumors had sustained proviral insertions near the Wnt-1 gene, the first member of this gene family. A biological assay for Wnt-1 was subsequently established using gene transfer experiments. This assay was used to show that certain mammary gland-derived cell lines can be morphologically transformed by Wnt-1. Direct evidence that Wnt-1 expression gives a strong growth stimulus to mammary cells came from transgenic mice carrying Wnt-1 linked to the MMTV promoter, which developed mammary hyperplasia and tumors. By infecting primary mammary cells with retroviruses expressing Wnt-1 and re-implantation of the infected cells, similar hyperplasia of the mammary gland were obtained. Additional experiments led to the identification of a Wnt-1 related oncogene activated by MMTV insertion, called Wnt-3.

The growth stimulus generated by the expression of Wnt-1 in the mammary gland implies that mammary cells are equipped with a Wnt receptor that becomes activated by the Wnt-1 protein, as well as the other signaling components. While neither Wnt-1 nor Wnt-3 are expressed in the normal mammary gland, at least 5 other Wnt genes are expressed during specific stages of mammary gland development, including during the rapid expansion of the pre-lactating gland or when the gland regresses.

The oncogenic action of Wnt-1 and Wnt-3 is best explained by their acting as ligands for Wnt receptors meant for other Wnt genes, and activating these receptors inappropriately. Alternatively, Wnt-1 and Wnt-3 may not activate these receptors but may interfere with a ligand-receptor interaction normally leading to regression of the gland.

The strong growth stimulus by oncogenic Wnt genes and the dynamic expression patterns of other Wnt genes in the mammary gland provide evidence that Wnt genes are important regulators of mammary gland growth. In analogy with the mouse, it is likely that some Wnt genes expressed during the normal cycles of growth of the mammary gland. In contrast to silent genes, genes that are expressed are candidates to become amplified, since the ensuing overexpression of those genes can give a selective advantage to cells even during the first rounds of amplification.

A survey of mouse mammary tumors identified one tumor where the mouse Wnt-2 gene was amplified and overexpressed whereas Wnt-2 had a low level of expression in the normal gland. Further, there was no evidence for insertion of MMTV near Wnt-2 in that tumor. This finding shows that Wnt genes are not necessarily activated only by MMTV, a relevant factor for human breast cancer since that disease has no viral etiology but is often characterized by gene amplification. In view of these observation, it is contemplated that inhibitors of Wnt/WntR interactions may be used to inhibit the growth and/or spread of breast and other types of cancer.

VII. Screening Methods

In view of the role of Wnt in cancer and other processes involving growth, development and proliferation (both normal and abnormal), it would be desirable to identify modulators of Wnt activity that affect the interactions of specific Wnt proteins with their receptors. Such modulators may, for example, inhibit the binding of Wnt to its receptor (e.g., by competitive or noncompetitive inhibition), or they may potentiate or stabilize the binding. The recognition that members of the frizzled family of proteins can act as receptors for the Wnt family of proteins enables a number of screening approaches to the isolation of such modulatory compounds that have heretofore not been possible.

Examples of such screening approaches include protein-protein binding assays in which the level of binding of Wnt to its receptor, or a biological consequence of such binding, is measured. The latter assay is exemplified in Example 4, where cells not normally expressing Wnt receptors are transformed with a Wnt receptor (in this case, Dfz2), and the effects of Wnt (in this case, Wg) on the cells are measured (in this case, by detecting levels of Arm). Such cells may be transformed with the Wnt receptor of choice (e.g., any of fz1, fz2, fz3, fz4, fz5, fz6, fz7 or fz8 receptors).

In Example 4, expression of Arm was detected using a Western blot method. Other methods may be employed which are more suitable for high throughput screening applications. For example, labelled anti-Arm antibodies may be used to directly visualize levels of Arm in multi-well format screen.

Alternatively, the assays may simply detect the degree of binding between Wnt ligands and Wnt receptors, and not the biological consequences of such binding. For example, cells expressing a selected Wnt receptor may be plated in the wells of a 96-well plate and contacted with a solution containing reporter-labeled Wnt (e.g., radiolabelled of fluorescently-tagged) in the presence and absence of a test compound (i.e., a putative modulator of Wnt/receptor interactions). The effect of the test compound on the extent of binding between Wnt and Wnt receptor is measured, and the compound is identified as effective if its effect on the extent of binding is above a threshold level (e.g., a several-fold difference in binding level between control and experimental samples) In one embodiment, the threshold is a 2-fold difference. In another embodiment, it is a 5-fold difference. In yet another it is a 10-fold or greater difference. The difference in binding in the presence and absence of an effective test compound is preferably statistically-significant, as determined by a standard statistical test.

It will be appreciated that the putative modulator compound can alternatively be added after the cells had been incubated with labelled Wnt. In a screen for inhibitors of binding, the system is assayed for a decrease in the signal reflecting bound labelled Wnt, or an increase in the signal reflecting labelled Wnt in solution.

Such a screen may also be employed to screen for potentiators of Wnt/receptor interactions. For example, test compounds may be added to the wells (either during or after incubation with labelled Wnt), and the wells then contacted with unlabeled Wnt. Test compounds in wells where the unlabelled Wnt is less effective at displacing the bound labelled Wnt are selected for more detailed examination of ability to potentiate Wnt/receptor binding.

Assays such as described above may also be used to determine the relationship between different Wnt proteins and different receptors. For example, the ligand concentration dependence of binding may be used in measurement of the relative affinities of selected Wnt receptors with selected ligands, and ligands with a selected affinity for the receptor can be examined further using, e.g., in vitro or in vivo assays. In this manner, one of skill in the art can identify which Wnt protein(s) is optimally paired with which receptor(s).

In cases where the Wnt ligand has been matched to a specific Wnt receptor (e.g., in the case of Wg and Dfz2), the receptor/ligand pair can be used in, e.g., screening applications. For example, the pair may be used in a binding assay to screen for compounds which are effective to modulate the binding of the specific ligand to its receptor. These methods enable the identification of compounds with two general types of activities: (i) those which act generally, e.g., on a class of Wnt/Wnt receptor pairs, to disrupt or facilitate binding, and (ii) those which act selectively disrupt or facilitate the binding between a selected Wnt ligand and its receptor, but not between other Wnt ligands and their receptors.

Compounds identified by one of the screens described herein may be further evaluated for efficacy using an in vitro assay such as described above. Further, such compounds may be tested in in vivo models employing Wnt/Wnt receptor interactions. For example, the compounds may be tested in a mouse mammary tumor model for effectiveness at inhibiting growth of mammary tumors.

VIII. Compounds Suitable for Screening

A variety of different compounds may be screened using methods of the present invention. They include peptides, macromolecules, small molecules, chemical and/or biological mixtures, and fungal, bacterial, or algal extracts. Such compounds, or molecules, may be either biological, synthetic organic, or even inorganic compounds, and may be obtained from a number of sources, including pharmaceutical companies and specialty suppliers of libraries (e.g., combinatorial libraries) of compounds.

In cases where an identified active compound is a peptide, the peptide may be utilized to design a peptoid mimetic and aid in the discovery of orally-active small molecule mimetics. Alternatively, the peptides themselves may be used as therapeutics.

Further, the structure of a bioactive polypeptide may be determined using, for example, NMR, and may be used to select the types of small molecules screened.

Methods of the present invention are well suited for screening libraries of compounds in multi-well plates (e.g., 96-well plates), with a different test compound in each well. In particular, the methods may be employed with combinatorial libraries. A variety of combinatorial libraries of random-sequence oligonucleotides, polypeptides, or synthetic oligomers have been proposed (Kramer, et al., 1993; Houghten, 1985, 1994; Houghten, et al., 1986, 1991, 1992; Ohlmayer, et al., 1993; Dooley, et al., 1993a-1993b; Eichler, et al., 1993; Pinilla, et al., 1992, 1993; Ecker, et al., 1993; and Barbas, et al., 1992). A number of small-molecule-libraries have also been developed (e.g., Bunin, et al., 1994; Bunin and Ellman, 1992; Virgilio and Ellman, 1994).

Combinatorial libraries of oligomers may be formed by a variety of solution-phase or solid-phase methods in which mixtures of different subunits are added stepwise to growing oligomers or parent compound, until a desired oligomer size is reached (typically hexapeptide or heptapeptide). A library of increasing complexity can be formed in this manner, for example, by pooling multiple choices of reagents with each additional subunit step (Houghten, et al., 1991).

Alternatively, the library may be formed by solid-phase synthetic methods in which beads containing different-sequence oligomers that form the library are alternately mixed and separated, with one of a selected number of subunits being added to each group of separated beads at each step (Furka, et al., 1991; Lam, et al., 1991, 1993; Zuckermann, et al., 1992; Sebestyen, et al., 1993).

The identity of library compounds with desired effects on the binding of a Wnt to a Wnt receptor can be determined by conventional means, such as iterative synthesis methods in which sublibraries containing known residues in one subunit position only are identified as containing active compounds.

IX. Pharmaceutical Preparations of Active Compounds

After identifying certain test compounds as potential WntR agonists or antagonists agents, the practitioner of the screening assay will typically continue to test the efficacy and specificity of the selected compounds both in vitro and in vivo. Whether for subsequent in vivo testing, or for administration to an animal as an approved drug, agents identified in the screening assay can be formulated in pharmaceutical preparations for in vivo administration to an animal, preferably a human.

The compounds selected in the screening assay, or a pharmaceutically acceptable salt thereof, may accordingly be formulated for administration with a biologically acceptable medium, such as water, buffered saline, polyol (for example, glyccrol, propylene glycol, liquid polyethylene glycol and the like) or suitable mixtures thereof. The optimum concentration of the active ingredient(s) in the chosen medium can be determined empirically, according to procedures well known to medicinal chemists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the compound, its use in the pharmaceutical preparation of the invention is contemplated.

Suitable vehicles and their formulation inclusive of other proteins are described, for example, in Gennaro, 1990. These vehicles include injectable “deposit formulations”. Based on the above, such pharmaceutical formulations include, although not exclusively, solutions or freeze-dried powders of the compound in association with one or more pharmaceutically acceptable vehicles or diluents, and contained in buffered media at a suitable pH and isosmotic with physiological fluids. In a preferred embodiment, the compound can be disposed in a sterile preparation for topical and/or systemic administration. In the case of freeze-dried preparations, supporting excipients such as, but not exclusively, mannitol or glycine may be used and appropriate buffered solutions of the desired volume will be provided so as to obtain adequate isotonic buffered solutions of the desired pH. Similar solutions may also be used for the pharmaceutical compositions in isotonic solutions of the desired volume and include, but not exclusively, the use of buffered saline solutions with phosphate or citrate at suitable concentrations so as to obtain at all times isotonic pharmaceutical preparations of the desired pH (for example, neutral pH).

The following examples illustrate but in no way are intended to limit the present invention.

### Materials and Methods

Unless otherwise indicated, restriction enzymes and DNA modifying enzymes were obtained from New England Biolabs (Beverly, Mass.) or Boehringer Mannheim (Indianapolis, Ind.). Nitrocellulose paper was obtained from Schleicher and Schuell (Keene, N.H.). Other chemicals were purchased from Sigma (St. Louis, Mo.) or United States Biochemical (Cleveland, Ohio). Unless otherwise specified, the experiments were performed using standard methods (Ausubel, et al., 1988; Sambrook, et al., 1989; Harlow, et al., 1988).

A. Buffers

Phosphate-buffered saline (PBS)

10× stock solution, 1 liter:

80 g NaCl

2 g KCl

11.5 g Na₂HPO4-7H₂O

2 g KH2P04

Working solution, pH 7.3:

137 mM NaCl

2.7 mM KCl

4.3 mM Na₂HPO₄-7H₂O

1.4 mM KH₂PO₄

### EXAMPLE 1

### Molecular Cloning of DFz2

Polymerase chain reaction (PCR; Mullis, 1987; Mullis, et al., 1987) primer pools YW157 and YW158 were designed based on sequences (SEQ ID NO:16, SEQ ID NO:17, respectively) conserved in Dfz1, Human frizzled 3 (Hfz3), Rat frizzled 1 (Rfz1) and Rat frizzled 2 (Rfz2). The primer pools were completely degenerate, that is, each possible codon of each amino acid in SEQ ID NO:16 and SEQ ID NO:17 was represented in the respective primer pool, with the exception that the wobble base of the 3′-most codon was not included in YW157. The primers were used to amplify Drosophila genomic DNA, resulting in an amplification product that, when sequenced, was found to contain a novel frizzled family member—Dfz2. The PCR product was used to isolate genomic clones of Dfz2 from an adult Drosophila genomic library (Maniatis, et al.) and cDNA clones from a 0-24 hr cDNA library.

The amino acid sequence of Dfz2 was compared to that of Dfz1 by aligning the sequences as shown in FIG. 1. Dfz2 and Dfz1 are 32% identical. Identical residues are indicated in the consensus and the conserved cysteine residues in the cysteine-rich domain are in bold-face. The sequence alignments were done using the “GENEWORKS” program.

Hydropathy values were calculated using the “MACVECTOR” 3.5 software according to the Kyte-Doolittle software and a window size of 15 amino acids.

### EXAMPLE 2

### In Situ RNA Hybridization

In situ hybridization experiments were performed to determine the pattern of Df z2 expression. Freshly dissected adult brains, whole embryos or heads were rapidly frozen in plastic molds placed on a dry ice/alcohol slurry and processed for sectioning as described previously (Cole, et al., 1990). ³⁵S-Labeled antisense riboprobes were prepared from linearized p“BLUESCRIPT” plasmid subclones using either T3 or T7 RNA polymerase. In situ hybridization was performed as described by Saffen, et al., and hybridized sections were exposed to X-ray film and digitized.

### EXAMPLE 3

### Expression of DFz2 During Drosophila Development

The expression pattern of DFz2 was assessed using Northern (RNA) blot analysis. Total RNA was isolated using the LiCl-Urea precipitation method (Auf fray and Rougeon, 1980). 30 microgram of RNA from each sample was resolved on a formaldehyde 1% agarose gel. The RNA was transferred to a nylon filter, cross-linked by UV irradiation and hybridized to a probe made by random priming Dfz2 or RP49 DNA fragments using standard methods (Sambrook, et al., 1989). In other experiments, Poly (A)⁺ RNA from various stages of Drosophila development was first selected from total RNA using the Invitrogen “FASTTRACK” 2.0 kit and 5 μg was loaded per lane.

Exemplary results are shown in FIG. 3. A 4.0 kb transcript was detected in embryonic stages 0-2; 2-3; 4-5; 9-12, first, second and third instar larvae and pupae. A transcript of similar size was observed in Drosophila clone-8 cells (cl-8), a cell line from imaginal discs previously shown to be responsive to Wg activity in vitro. Drosophila Schneider 2 (S2) cells, which do not respond to Wg, did not contain detectable DFz2 transcripts. The blot was also probed for expression of the ribosomal protein RP49 (O'Connell and Rosbash, 1994, lower panel) as a control for RNA integrity and loading.

### EXAMPLE 4

### Transfection of DFz2 in S2 Cells Confers a Response to Wq Protein

S2 cells were evaluated for Dfz2 expression because the cells are known not to respond to Wg (Yanagawa, et al., 1995). Since, as described above, the native cells did not express Dfz2, they were used in Dfz2 transfection experiments to determine whether expression of Dfz2 would confer sensitivity to Wg.

An expression vector containing DFz2 coding sequences under the control of a metal-inducible metallothionein promoter was used to transfect S2 cells using standard methods. Stable cell lines were derived by selection in hygromycin and tested for Dfz2 expression. In cells grown in the absence of inducers, a baseline level of expression was detected with an antiserum to Dfz2. Induction of the metallothionein promoter resulted in increased levels of expression.

Sensitivity of the Dfz2-transfected S2 cells to Wg protein was assessed by measuring the levels of armadillo (Arm) protein in response to Wg application. In intact Drosophila embryos and in clone-8 cells, Arm protein migrates in two different forms, differing from each other in phosphorylation. When these cells are incubated in the presence of soluble Wg protein, the level of the faster migrating (non-phosphorylated) form increases (Peifer, et al., 1994; Riggleman, et al., 1990; Van Leeuwen, et al., 1994). This increase can be detected using a standard Western blot assay as described below.

Conditioned medium containing Wg protein was produced by subjecting S2HSwg cells to heat-shock for 30 minutes at 37° C., allowing the cells to recover for 30 minutes at 25° C., and resuspending them in S2 medium without fetal calf serum (FCS). The cells were incubated for 3 hrs to allow secretion of proteins into the medium, after which they were removed by centrifugation (10 min., 2000×g and 1 hr, 100,000 xg, respectively) The conditioned media were concentrated 12-fold (“CENTRIPREP30”, Amicon) and used in the experiments as follows.

Clone 8, untransformed S2, and Dfz-transformed S2 (S2Dfz2) cells were incubated for 2 hrs in 6-well dishes in either normal concentrated medium or in concentrated medium from S2 cells producing Wg. Overexpression of the Dfz2 gene (under control of the metallothionein promoter) was induced by culturing S2Dfz2 and S2 control cells in S2 medium containing 0.5 mM CuSO₄ for 5 hrs prior to the incubation with the conditioned media.

The target cells were lysed in lysis buffer (50 mM Tris, pH 7.5, 150 mM NaCl, 1% Nonidet-P40, 5 mM EDTA) supplemented with 20 μg leupeptin, 100 μg aprotinin and 180 μg PMSF per ml. The extracts were subjected to electrophoresis and Western blotting. Blots were stained in Ponceau Red to evaluate equal loading of total protein and transfer, and then incubated overnight in blocking buffer with monoclonal anti-arm antibody 7A1 at a 1:1000 dilution or rat-polyclonal anti-α-catenin antibody DCAT-1 (Oda, et al., 1993), diluted 1:1000. The blots were washed three times for 15 min each in TBST and incubated for 1 hr with horseradish peroxidase conjugated secondary antibodies (Biorad) diluted 1:20,000 in blocking buffer.

Incubation of DFz2-transfected S2 cells (but not untransfected S2 cells) in the presence of soluble Wg protein resulted in an increase in the level of Arm protein similar to that observed in Drosophila embryos and clone-8 cells. Exemplary results are shown in FIG. 4. Addition of Wg (wingless) results in increased signal intensity of the armadillo band. No such effect is observed with untransfected S2 cells. However, all four independent Dfz2-transfected S2 cell lines, derived from two separate transfections, showed increased armadillo signal in response to Wg (two of the four are shown). Further induction of Dfz2 expression by copper sulphate in the transfected cells led to a slight decrease in the response to Wg. As a control for equal loading, the blots were stripped and incubated with an antiserum against a-catenin (lower panel).

### EXAMPLE 5

### Wg Protein Binds to Dfz2 Transfected Cells

The results described in Example 4 showed that Dfz2 acts as a signal transducing molecule for Wg, suggesting that it is a receptor for Wg. Immunohistochemical analyses were performed to determine whether Wg was capable of binding to the Dfz2-transfected cells.

Nontransfected Sneider 2 (S2) cells and S2 cells expressing Dfz2 were washed twice in PBS and incubated with 1.5 ml of medium alone or 1.5 ml of a 10× concentrated stock of Wg conditioned medium at 4° C. for 3 hours. After three 10 minute washes with PBS, the cells were fixed in 2% methanol-free formaldehyde (Polysciences, Inc) for 15 minutes at room temperature. Following three more 10 minute washes with PBS, affinity purified Wg antibody at 1/25 and 5% donkey serum were added to the cells in PBS and incubated overnight at 4° C.

The antiserum was affinity-purified using a bacterial fusion protein containing a domain unique to Wg (the Wg insert—an 85 amino acid sequence not found in any wg orthologs). Previous experiments have indicated that this domain is dispensable for Wg activity, that it probably does not participate in the interactions between Wg and its receptor.

Following 3 additional 10 minute washes, fluorescent-labeled cy3 secondary antibody, donkey anti-rabbit (Sigma), at 1/100 and 5% donkey serum were added to the cells for 1 hour at room temperature. The cells were then washed 3 more times in PBS and mounted in Vectashield mounting medium (Vector).

Confocal images were collected with a Bio-Rad MRC 1000 confocal laser attached to a Zeiss Axio scope microscope. Exemplary images are shown in FIGS. 5A-5F. Normal and transfected cells were incubated with either normal S2 medium (FIG. 5A) or concentrated conditioned medium from S2 cells producing Wg (FIGS. 5B, 5C, 5D, 5E, 5F). Dfz2-transfected S2 cells stained brightly in approximately 80% of the cells when incubated with Wg and the antiserum (FIG. 5D) whereas Dfz2-transfected cells in the absence of Wg protein (FIG. 5A) as well as non transfected S2 cells (FIG. 5B) showed only some spots of background staining. The positive staining was not uniform over the cell surface but punctate and may reflect clustering of receptor complexes.

The ability of Wg to bind was also tested in heterologous cells (human 293 cells) transiently-transfected with Dfz2. In view of high background binding observed in initial experiments, the transiently-transfected 293 cells were preincubated with chlorate, which inhibits sulfation of proteins and glucosaminoglycans, and with heparatinase, to remove heparin-like molecules. This pre-treatment significantly lowered the background binding (presumably due to Wg binding to extracellular matrix; FIG. 5E). As shown in FIG. 5F, about 10-20% of the transfected cells remained positive, similar to the transfection efficiency of 293 cells. Since 293 cells are of human origin, these results strongly suggest that Wg binds to Dfz2 itself, rather than to a molecule whose expression is induced by Dfz2.

In contrast to the positive staining patterns observed with Dfz2-transfected cells, no staining was detected in S2 cells expressing Notch (FIG. 5C). Notch is a protein that has been previously proposed to act as a receptor for Wg (Couso and Arias, 1994).

The above results taken together indicate that Wg protein can specifically bind to cells expressing Dfz2, and that this binding is not likely due to clonal variation.

### EXAMPLE 6

Binding of Metabolically-Labeled Wg Protein to a Dfz-2/IgG Fusion Protein

The binding of Wg protein to Dfz2 itself was also assayed using a fusion protein containing the cysteine-rich amino-terminal domain of Dfz2, linked to the constant domain of human IgG. The fusion protein or IgG control was added to conditioned medium from normal S2 cells, or S2 cells producing Wg (HS-wg/S2), which had been metabolically-labeled with [³⁵S] cysteine and methionine.

The fusion proteins and possible complexes were then retrieved by adding sepharose-ProteinA beads and analyzed by gel electrophoresis and fluorography. FIG. 6 shows that the Dfz2 fusion protein, but not the control IgG, selectively binds to labeled proteins of 52 kD, the size of the mature Wg protein. Normal S2 cells did not produce Dfz-2 binding proteins.

While the invention has been described with reference to specific methods and embodiments, it is appreciated that various modifications and changes may be made without departing from the invention.

It is claimed:

1. An isolated nucleic acid molecule encoding a Wnt receptor having an amino acid sequence that is greater than about 90% identical to the amino acid sequence of a Wnt receptor selected from the group consisting of Dfz2, Mfz3, Mfz4, Hfz5, Mfz6, Mfz7, Mfz8, and Cfz1.

2. A nucleic acid molecule of claim 1 encoding a Wnt receptor having an amino acid sequence that is greater than about 90% identical to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14 and SEQ ID NO:16.

3. A nucleic acid molecule of claim 2 encoding a Wnt receptor having an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14 and SEQ ID NO:16.

4. A nucleic acid molecule of claim 3 encoding a Wnt receptor having the amino acid sequence represented as SEQ ID NO:2.

5. A nucleic acid molecule of claim 1, wherein said molecule encodes a human Wnt receptor.

6. An isolated Wnt receptor polypeptide having an amino acid sequence that is greater than about 90% identical to the amino acid sequence of a Wnt receptor selected from the group consisting of Dfz2, Mfz3, Mfz4, Hfz5, Mfz6, Mfz7, Mfz8, and Cfz1.

7. A polypeptide of claim 6, wherein said polypeptide is a human Wnt receptor polypeptide.

8. A polypeptide of claim 6 having an amino acid sequence that is greater than about 90% identical to an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14 and SEQ ID NO:16.

9. A polypeptide of claim 8 having an amino acid sequence selected from the group consisting of SEQ ID NO:2, SEQ ID NO:4, SEQ ID NO:6, SEQ ID NO:8, SEQ ID NO:10, SEQ ID NO:12, SEQ ID NO:14 and SEQ ID NO:16.

10. A polypeptide of claim 9 having the amino acid sequence represented as SEQ ID NO:2.

11. A method of identifying a compound capable of affecting binding of a Wnt polypeptide to a Wnt receptor (WntR) polypeptide, comprising
contacting such a WntR with a selected Wnt polypeptide, in the presence and absence of a test compound,
measuring the effect of the test compound on the extent of binding between Wnt and said WntR, and
identifying said compound as effective to alter binding of a Wnt polypeptide to a WntR polypeptide if its measured effect on the extent of binding is above a threshold level.

12. The method of claim 11, wherein said threshold is a 2-fold or greater inhibition of binding.

13. The method of claim 11, wherein said threshold is a 2-fold or greater potentiation of binding.

14. The method of claim 11, wherein said Wnt polypeptide is wingless (Wg).

15. The method of claim 11, wherein said WntR polypeptide is Dfz2.

16. The method of claim 15, wherein said WntR polypeptide has the amino acid sequence represented as SEQ ID NO:2.

17. The method of claim 11, wherein said test compound is effective to inhibit binding between the Wnt polypeptide and the WntR polypeptide.

18. The method of claim 11, wherein said test compound is effective to displace the Wnt polypeptide from the WntR polypeptide.

19. The method of claim 11, wherein said WntR polypeptide is expressed on the surface of a cell transformed with an expression vector encoding said receptor.

20. The method of claim 19, wherein said cell is a Drosophila Sneider 2 (S2) cell and said expression vector encodes the WntR polypeptide Dfz2.

21. The method of claim 11, wherein said WntR polypeptide is an N-terminal portion of a full-length WntR polypeptide, said portion including the cysteine-rich amino-terminal domain.

22. The method of claim 21, wherein said portion is a first part of a fusion protein.

23. The method of claim 22, wherein said fusion protein further includes a second portion, said second portion containing the constant domain of human IgG.

24. The method of claim 11, further comprising preparing a pharmaceutical preparation of a compound identified as effective to alter binding of a Wnt polypeptide to a WntR polypeptide.
